(19) United States
(12) Patent Application Publication
(10) Pub. No.: US 2018/0214053 A1
(43) Pub. Date: Aug. 2, 2018

(54) COCHLEOPALPEBRAL REFLEX TEST

(71) Applicant: MARCELINO COVARRUBIAS, GOMEZ PAL (MX)

(72) Inventor: MARCELINO COVARRUBIAS, GOMEZ PAL (MX)

(21) Appl. No.: 15/383,364

(22) Filed: Dec. 19, 2016

(51) Int. Cl. A61B 5/12; H03F 3/45; A61B 5/024; A61B 8/08

(52) U.S. Cl. CPC A61B 5/125; H03F 3/45071; H03F 2203/45596; A61B 8/0866; H03F 2203/45544; A61B 5/02411

(57) ABSTRACT

The Cochleopalpebral reflex test is an electroacoustic transducer that emits a harmless sound stimulus that induces the cochlea palpebral reflex and the subsequent variation in the heart rate of the fetus that is detected as described in the prior paragraph. The Cochleopalpebral reflex test consists of a housing, a transducer, an amplifier, and a signal source. The transducer and amplifier are contained within the housing. The signal source is an externally generated electrical signal of a previously determined frequency. The previously determined frequency is in the audible range of humans. The electrical signal of a previously determined frequency is amplified by the amplifier and is converted into acoustic energy and introduced to the fetus through the transducer.

### CROSS REFERENCES TO RELATED APPLICATIONS

Not Applicable

### STATEMENT REGARDING FEDERALLY SPONSORED RESEARCH

Not Applicable

### REFERENCE TO APPENDIX

Not Applicable

### BACKGROUND OF THE INVENTION

The present invention relates to the field of medical and veterinary science, more specifically, a diagnostic tool using ultrasonic, sonic, or infrasonic waves.

A prenatal diagnosis of deafness is primordial for the best outcome of every prenatal case. The sooner one can stimulate the central auditory system of a human, the easier it is to learn a language naturally. Currently, the earliest diagnosis for deafness is targeted to newborns. This method of diagnosis has difficulties including: 1) a lack of broad accessibility to diagnosis for deafness for infants; and, 2) current screening techniques cannot detect cases with auditory neuropathy which represents 11% of the total cases of deafness reported in scientific literature.

Clearly, a readily accessible test for the early diagnosis of deafness that is expanded to detect auditory neuropathy would be of benefit.

### SUMMARY OF INVENTION

The Cochleopalpebral reflex test offers a more practical and accurate solution for the early diagnosis of deafness. The Cochleopalpebral reflex is further capable of testing for auditory neuropathy. The Cochleopalpebral reflex test uses tools commonly available within the currently existing infrastructures that provide a universal health service to the population.

The Cochleopalpebral reflex test is a prenatal deafness detection system. The Cochleopalpebral reflex test is based on the detection of the cochlea palpebral reflex. Specifically, the Cochleopalpebral reflex test detects changes in the motor responses of a fetus to a previously determined sound stimulus. The Cochleopalpebral reflex test comprises of an emission of a sound stimulus produced by the Cochleopalpebral reflex test during an ultrasound exploration. The motor response is measured as an increase in the fetal heart rate frequency. The measurement of the fetal heart rate is commonly done in conjunction with a prenatal ultrasound imaging. The motor response can be further confirmed by monitoring the orbicularis oculi of the fetus. This method is possible nowadays due to the development of technologies in this field that allow the scanning of the fetus face during the ultrasound exploration. This allows us to look for the cochlea palpebral reflex in the orbicularis oculi and through this, find out further confirm that the auditory central system is developing correctly.

The Cochleopalpebral reflex test is an electroacoustic transducer that emits a harmless sound stimulus that induces the cochlea palpebral reflex and the subsequent variation in the heart rate of the fetus that is detected as described in the prior paragraph. The Cochleopalpebral reflex test consists of a housing, a transducer, an amplifier, and a signal source. The transducer and amplifier are contained within the housing. The signal source is an externally generated electrical signal of a previously determined frequency. The previously determined frequency is in the audible range of humans. The electrical signal of a previously determined frequency is amplified by the amplifier and is converted into acoustic energy and introduced to the fetus through the transducer.

These together with additional objects, features and advantages of the Cochleopalpebral reflex test will be readily apparent to those of ordinary skill in the art upon reading the following detailed description of the presently preferred, but nonetheless illustrative, embodiments when taken in conjunction with the accompanying drawings.

In this respect, before explaining the current embodiments of the Cochleopalpebral reflex test in detail, it is to be understood that the Cochleopalpebral reflex test is not limited in its applications to the details of construction and arrangements of the components set forth in the following description or illustration. Those skilled in the art will appreciate that the concept of this disclosure may be readily utilized as a basis for the design of other structures, methods, and systems for carrying out the several purposes of the Cochleopalpebral reflex test.

It is therefore important that the claims be regarded as including such equivalent construction insofar as they do not depart from the spirit and scope of the Cochleopalpebral reflex test. It is also to be understood that the phraseology and terminology employed herein are for purposes of description and should not be regarded as limiting.

### BRIEF DESCRIPTION OF DRAWINGS

The accompanying drawings, which are included to provide a further understanding of the invention are incorporated in and constitute a part of this specification, illustrate an embodiment of the invention and together with the description serve to explain the principles of the invention. They are meant to be exemplary illustrations provided to enable persons skilled in the art to practice the disclosure and are not intended to limit the scope of the appended claims.

FIG. 1 is a perspective view of an embodiment of the disclosure.

FIG. 2 is a top view of an embodiment of the disclosure.

FIG. 3 is a cross-sectional view of an embodiment of the disclosure across 3-3 as shown in FIG. 2.

FIG. 4 is a schematic view of an embodiment of the disclosure.

FIG. 5 is a wiring diagram of an embodiment of the disclosure.

### DETAILED DESCRIPTION OF THE EMBODIMENT

The following detailed description is merely exemplary in nature and is not intended to limit the described embodiments of the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the appended claims. Furthermore, there is no intention to be bound by any expressed or implied theory presented in the preceding technical field, background, brief summary or the following detailed description.

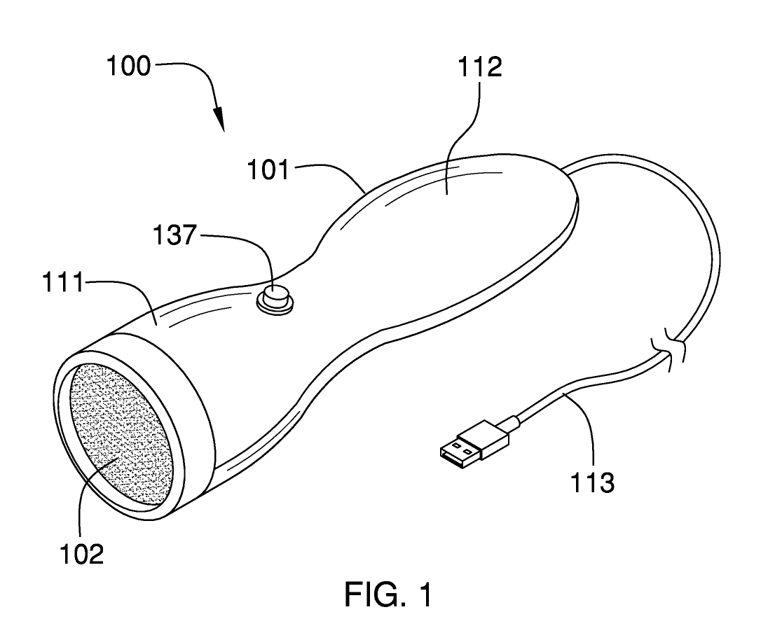

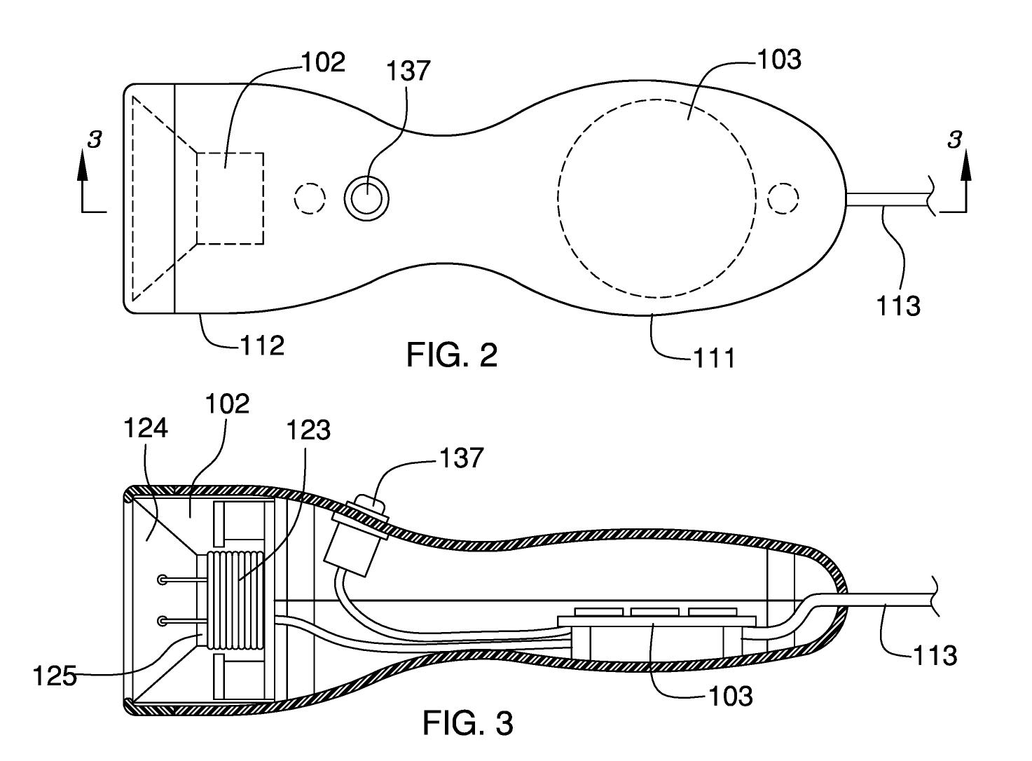

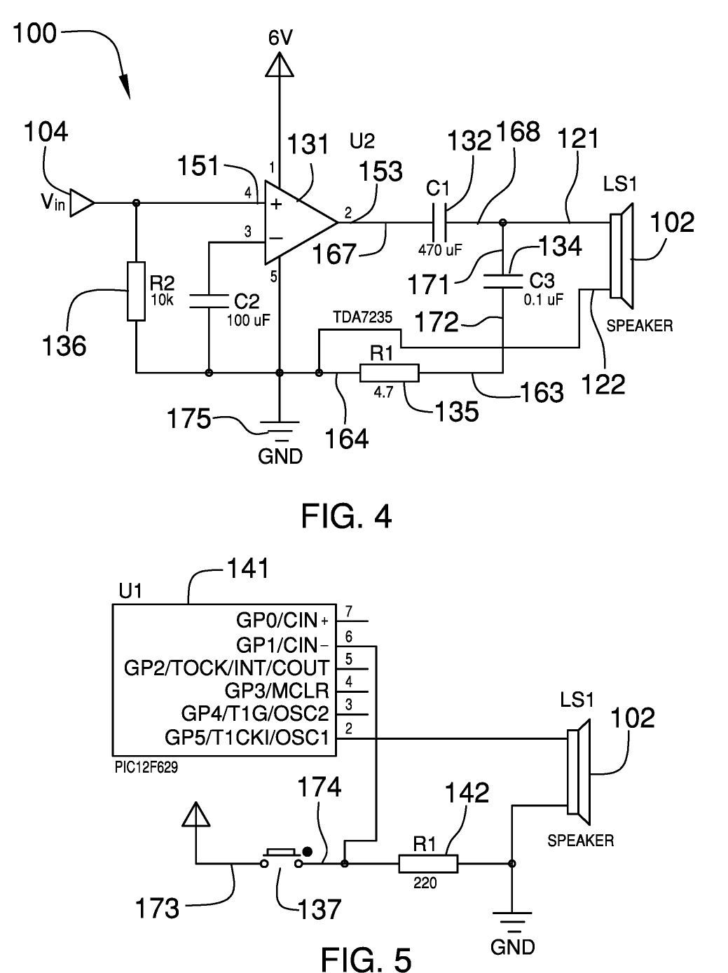

Detailed reference will now be made to one or more potential embodiments of the disclosure, which are illustrated in FIGS. 1 through 5.

The Cochleopalpebral reflex test 100 (hereinafter invention) is an electroacoustic transducer 102 that emits a harmless sound stimulus that induces the cochlea palpebral reflex and the subsequent variation in the heart rate of the fetus that is detected as described elsewhere in this disclosure. The invention 100 comprises a housing 101, a transducer 102, an amplifier circuit 103, and a signal source 104. The transducer 102 and amplifier circuit 103 are contained within the housing 101. The signal source 104 is an externally generated electrical signal of a previously determined frequency. The previously determined frequency is in the audible range of humans. The signal source 104 is amplified by the amplifier circuit 103 and is converted into acoustic energy and introduced to the fetus through the transducer 102.

The housing 101 is a rigid handheld casing within which the transducer 102 and the amplifier circuit 103 are contained. The housing 101 comprises a grip 111, a bell 112, and a cable 113. The grip 111 is a hollow shaft structure that is used as a handle to manipulate the invention 100 during use. In the first potential embodiment of the disclosure, the grip 111 contains the amplifier circuit 103. The bell 112 is a cone shaped structure that contains the transducer 102. The base of the cone structure that forms the bell 112 is open such that the acoustic energy generated by the transducer 102 will escape the bell 112. The cable 113 is an electrical conductor that transmits the signal source 104 to the amplifier circuit 103.

The transducer 102 is a device that converts an electrical signal into acoustic energy. In the first potential embodiment of the disclosure, the transducer 102 is a readily and commercially available speaker. The use of speakers in electrical circuits are well known and documented in the electrical arts. The transducer 102 comprises a first lead 121, a second lead 122, a coil 123, a diaphragm 124, and a magnet 125. The coil 123 is a wire coil that is used to generate a magnetic field that is used to move the magnet 125. The diaphragm 124 is a membrane which is moved by the magnet 125 within the coil 123. The movement of the diaphragm 124 generates the acoustic waves necessary to create audible sounds. The magnet 125 is a commercially available magnet that is moved by the coil 123. The magnet 125 is attached to the diaphragm 124 such that movements of the magnet 125 move the diaphragm 124.

The amplifier circuit 103 is an electrical circuit. The purpose of the amplifier circuit 103 is to increase the power of the signal source 104 such that the signal source 104 can be converted into audible acoustic energy by the transducer 102. The amplifier circuit 103 comprises an operational amplifier 131, a first capacitor 132, a second capacitor 133, a third capacitor 134, a first resistor 135, a second resistor 136, and a switch 137. The operational amplifier 131 is further defined with a positive input 151, an inverting input 152, and an output 153. The first capacitor 132 is further defined with a seventh lead 167 and an eighth lead 168. The second capacitor 133 is further defined with a ninth lead 169 and a tenth lead 170. The third capacitor 134 is further defined with an eleventh lead 171 and a twelfth lead 172. The first resistor 135 is further defined with a third lead 163 and a fourth lead 164. The second resistor 136 is further defined with a fifth lead 165 and a sixth lead 166. The switch 137 is further defined with a thirteenth lead 173 and a fourteenth lead 174.

The operational amplifier 131 is a readily and commercially available differential amplifier. The positive input 151 is a first signal input port of the operational amplifier 131. The inverting input 152 is a second signal input port of the operational amplifier 131. The inverting input 152 inverts the voltage of the applied to the inverting input 152 such that when the voltages applied to the positive input 151 and the inverting input 152 are combined the difference between the two signals are presented to the operational amplifier 131 for amplification. The output 153 is the port of the operational amplifier 131 from which the amplified signal of the operational amplifier 131 is accessed. Methods to incorporate an operational amplifier 131 into electrical circuits are well known and documented in the electrical arts.

The first capacitor 132 is a readily and commercially available capacitor. The first capacitor 132 is a filter that passes the sound stimulus from the operational amplifier 131 to the transducer 102. In the first potential embodiment of the disclosure, the first capacitor 132 is optimized to pass a sound stimulus of a frequency of 500 Hz. The second capacitor 133 is a readily and commercially available capacitor. The second capacitor 133 attaches the inverting input 152 of the operational amplifier 131 to the ground 175. The purpose of the second capacitor 133 is to reduce signal noise generated by the operational amplifier 131. The third capacitor 134 is a readily and commercially available capacitor. The purpose of the third capacitor 134 is to shunt high frequency noise away from the transducer 102 during use of the invention 100.

The first resistor 135 is a readily and commercially available resistor. The first resistor 135 limits current flow through the amplifier circuit 103. The second resistor 136 is a readily and commercially available resistor. The purpose of the second resistor 136 is to present the voltage of the externally provided signal source 104 such that the externally provided signal source 104 can be detected by the positive input 151 of the operational amplifier 131.

The switch 137 is a readily and commercially available momentary switch. As shown most clearly in FIG. 5, the switch 137 is used to initiate and discontinue the generation of the sound stimulus. As shown in FIG. 1, the switch 137 is mounted on the exterior of the grip 111 of the housing 101 such that the switch 137 can be manually manipulated during the use of the invention 100.

In a second potential embodiment of the disclosure, the amplifier circuit 103 comprises a TDA 7235 integrated circuit 141 and the third resistor 142. The TDA 7235 integrated circuit 141 is an integrated circuit that performs the functions of the amplifier circuit 103. The third resistor 142 is a readily and commercially available resistor. The purpose of the third resistor 142 is to carry an externally provided voltage such that the externally provided voltage can be detected by the TDA 7235 integrated circuit 141.

The signal source 104 is an externally provided voltage. When the amplitude of the signal source 104 is graphed against time the voltage of the signal source 104 forms a sine wave of a predetermined frequency. In the first potential embodiment of the disclosure, the predetermined frequency is 500 Hz. In the first potential embodiment of the disclosure, the signal source 104 is generated by a computer. The selected cable 113 is a readily and commercially available USB cable.

The amplifier circuit 103 is assembled as describe in this paragraph. Throughout this disclosure, the ground 175 refers to a common voltage reference point that is used throughout the amplifier circuit 103 and by the transducer 102 as a common current return path. The signal source 104 attaches to the positive input 151 of the operational amplifier 131 and attaches to the fifth lead 165 of the switch 137. The sixth lead 166 of the second resistor 136 attaches to the ground 175. The ninth lead 169 of the second capacitor 133 attaches to the inverting input 152 of the operational amplifier 131. The tenth lead 170 of the second capacitor 133 attaches to the ground 175. The output 153 of the operational amplifier 131 attaches to the seventh lead 167 of the first capacitor 132. The eighth lead 168 of the first capacitor 132 attaches to the first lead 121 of the transducer 102. The eighth lead 168 of the first capacitor 132 attaches to the eleventh lead 171 of the third capacitor 134. The twelfth lead 172 of the third capacitor 134 attaches to the third lead 163 of the first resistor 135. The fourth lead 164 of the first resistor 135 attaches to the ground 175. The second lead 122 of the transducer 102 attaches to the ground 175.

The following definitions were used in this disclosure:

Amplifier: As used in this disclosure, an amplifier refers to an electronic component that increases voltage, current, or power of an input signal. Specifically, within this disclosure, an amplifier refers to a differential amplifier. A differential amplifier is a device two input electrical device with a single output. A difference amplifier amplifies the voltage difference between the two inputs.

Cable: As used in this disclosure, a cable is a collection of insulated wires covered by a protective casing that is used for transmitting electricity or telecommunication signals.

Cochleopalpebral Reflex: As used within this disclosure, the cochlea palpebral reflex is an instinctive reflex in humans to an intense sound. While the cochlea palpebral reflex initiates many responses to the intense sounds including, but not limited to, a contraction of the orbicularis oculi muscle for which the cochlea palpebral reflex is named. The cochlea palpebral reflex is what makes people blink in response to loud noises.

Exterior: As used in this disclosure, the exterior is use as a relational term that implies that an object is not contained within the boundary of a structure or a space.

Handheld: As used in this disclosure, when referring to an item or device, handheld means that the item or device is small and light enough to be operated while a person holds the item or device in their hands.

Handle: As used in this disclosure, a handle is an object by which a tool, object, or door is held or manipulated with the hand.

Housing: As used in this disclosure, a housing is a rigid casing that encloses and protects one or more devices.

Interior: As used in this disclosure, the interior is use as a relational term that implies that an object is contained within the boundary of a structure or a space.

Lead: As used in this disclosure, a lead is a conductor that is physically used to electrically connect an electrical component into a larger circuit assembly.

Magnet: As used in this disclosure, a magnet is an ore, alloy, or other material that has its component atoms arranged so the material exhibits properties of magnetism such as attracting other iron-containing objects or aligning itself in an external magnetic field.

Momentary Switch: As used in this disclosure, a momentary switch is a biased switch in the sense that the momentary switch has a baseline position that only changes when the momentary switch is actuated (for example when a pushbutton switch is pushed). The momentary switch then returns to the baseline position once the actuation is completed. This baseline position is called the “normal” position. So for example, a “normally open” momentary switch interrupts (open) the electric circuit in the baseline position and completes (closes) the circuit when the momentary switch is activated. Similarly, a “normally closed” momentary switch will complete (close) an electric circuit in the baseline position and interrupt (open) the circuit when the momentary switch is activated.

Orbicularis Oculi: As used in this disclosure, the orbicularis oculi is a muscle in the human body that opens and closes an eyelid. The orbicularis oculi is also referred to as the orbicularis palpebrarum

Speaker: As used in this disclosure, a speaker is an electrical device that converts an electrical signal into an audible sound.

Switch: As used in this disclosure, a switch is an electrical device that starts and stops the flow of electricity through an electric circuit by completing or interrupting an electric circuit. The act of completing or breaking the electrical circuit is called actuation. Completing or interrupting an electric circuit with a switch is often referred to as closing or opening a switch respectively. Completing or interrupting an electric circuit is also often referred to as making or breaking the circuit respectively.

Transducer: As used in this disclosure, a transducer is a device that converts a physical quantity, such as pressure or brightness into an electrical signal or a device that converts an electrical signal into a physical quantity.

USB: As used in this disclosure, USB is an acronym for Universal Serial Bus which is an industry standard that defines the cables, the connectors, the communication protocols and the distribution of power required for interconnections between electronic devices. The USB standard defines several connectors including, but not limited to, USB-A, USB-B, mini-USB, and micro USB connectors.

With respect to the above description, it is to be realized that the optimum dimensional relationship for the various components of the invention described above and in FIGS. 1 through 5 include variations in size, materials, shape, form, function, and manner of operation, assembly and use, are deemed readily apparent and obvious to one skilled in the art, and all equivalent relationships to those illustrated in the drawings and described in the specification are intended to be encompassed by the invention.

It shall be noted that those skilled in the art will readily recognize numerous adaptations and modifications which can be made to the various embodiments of the present invention which will result in an improved invention, yet all of which will fall within the spirit and scope of the present invention as defined in the following claims. Accordingly, the invention is to be limited only by the scope of the following claims and their equivalents.

What is claimed is:

1. A medical diagnostic tool comprising
a housing, a transducer, an amplifier circuit, and a signal source;
wherein the transducer and amplifier circuit are contained within the housing;
wherein the signal source is an externally generated electrical signal of a previously determined frequency;
wherein the previously determined frequency is in the audible range of humans;
wherein the medical diagnostic tool induces the cochlea palpebral reflex in a fetus;
wherein the signal source is amplified by the amplifier circuit and is converted into acoustic energy using the transducer;
wherein the acoustic energy is introduced to the fetus through the transducer.

2. The medical diagnostic tool according to claim 1
wherein the housing is a rigid handheld casing within which the transducer and the amplifier circuit are contained;
wherein the housing comprises a grip, a bell, and a cable;
wherein the grip is a hollow shaft structure;
wherein the bell is a cone shaped structure that contains the transducer;
wherein the bell is attached to the grip;
wherein the cable is an electrical conductor.

3. The medical diagnostic tool according to claim 2 wherein the base of the cone structure that forms the bell is open such that the acoustic energy generated by the transducer will escape the bell.

4. The medical diagnostic tool according to claim 3
wherein the transducer is a device that converts an electrical signal into acoustic energy;
wherein the transducer is a speaker;
wherein the transducer is further defined with a first lead and a second lead.

5. The medical diagnostic tool according to claim 4
wherein the amplifier circuit is an electrical circuit;
wherein the amplifier circuit is to increase the power of the signal source such that the signal source can be converted into audible acoustic energy by the transducer.

6. The medical diagnostic tool according to claim 5
wherein the amplifier circuit comprises an operational amplifier, a first capacitor, a second capacitor, a third capacitor, a first resistor, a second resistor, and a switch;
wherein the operational amplifier, the first capacitor, the second capacitor, the third capacitor, the first resistor, the second resistor, and the switch are electrically interconnected;
wherein the operational amplifier is further defined with a positive input, an inverting input, and an output;
wherein the first capacitor is further defined with a seventh lead and an eighth lead;
wherein the second capacitor is further defined with a ninth lead and a tenth lead;
wherein the third capacitor is further defined with an eleventh lead and a twelfth lead;
wherein the first resistor is further defined with a third lead and a fourth lead;
wherein the second resistor is further defined with a fifth lead and a sixth lead;
wherein the switch is further defined with a thirteenth lead and a fourteenth lead.

7. The medical diagnostic tool according to claim 6 wherein the operational amplifier is a differential amplifier.

8. The medical diagnostic tool according to claim 7
wherein the first capacitor is a capacitor;
wherein the first capacitor is a filter that passes the sound stimulus from the operational amplifier to the transducer.

9. The medical diagnostic tool according to claim 8 wherein in the first capacitor is selected to pass a sound stimulus of a frequency of 500 hz.

10. The medical diagnostic tool according to claim 9
wherein the second capacitor is a capacitor;
wherein the second capacitor reduces signal noise generated by the operational amplifier.

11. The medical diagnostic tool according to claim 10
wherein the third capacitor is a capacitor;
wherein the third capacitor shunts high frequency noise away from the transducer during use of the medical diagnostic tool.

12. The medical diagnostic tool according to claim 11
wherein the first resistor is a resistor;
wherein the first resistor limits current flow through the amplifier circuit.

13. The medical diagnostic tool according to claim 12
wherein the second resistor is a resistor;
wherein the second resistor presents the voltage of the signal source to the positive input of the operational amplifier.

14. The medical diagnostic tool according to claim 13
wherein the switch is a momentary switch;
wherein the switch initiates and discontinues the generation of the sound stimulus;
wherein the switch is mounted on the exterior of the grip of the housing such that the switch can be manually manipulated during the use of the medical diagnostic tool.

15. The medical diagnostic tool according to claim 14
wherein the signal source is an externally provided voltage;
wherein when the amplitude of the signal source is graphed against time the voltage of the signal source forms a sine wave of a predetermined frequency;
wherein in the first potential embodiment of the disclosure, the predetermined frequency is 500 hz;
wherein in the first potential embodiment of the disclosure, the signal source is generated by a computer;
wherein the selected cable is a readily and commercially available USB cable.

16. The medical diagnostic tool according to claim 14
wherein the signal source attaches to the positive input of the operational amplifier and attaches to the fifth lead of the switch;
wherein the sixth lead of the second resistor attaches to the ground;
wherein the ninth lead of the second capacitor attaches to the inverting input of the operational amplifier;
wherein the tenth lead of the second capacitor attaches to the ground;
wherein the output of the operational amplifier attaches to the seventh lead of the first capacitor;
wherein the eighth lead of the first capacitor attaches to the first lead of the transducer;
wherein the eighth lead of the first capacitor attaches to the eleventh lead of the third capacitor;
wherein the twelfth lead of the third capacitor attaches to the third lead of the first resistor;
wherein the fourth lead of the first resistor attaches to the ground;
wherein the second lead of the transducer attaches to the ground.

17. The medical diagnostic tool according to claim 5
wherein the amplifier circuit comprises a TDA 7235 integrated circuit and the third resistor;
wherein the TDA 7235 integrated circuit is an integrated circuit that performs the functions of the amplifier circuit;
wherein the third resistor is a resistor.

18. The medical diagnostic tool according to claim 17
wherein the third resistor presents an externally provided voltage such that the externally provided voltage can be detected by the TDA 7235 integrated circuit.

19. The medical diagnostic tool according to claim 18
wherein the switch is a momentary switch;
wherein the switch initiates and discontinues the generation of the sound stimulus;
wherein the switch is mounted on the exterior of the grip of the housing such that the switch can be manually manipulated during the use of the medical diagnostic tool.

20. The medical diagnostic tool according to claim 19
wherein the signal source is an externally provided voltage;
wherein when the amplitude of the signal source is graphed against time the voltage of the signal source forms a sine wave of a predetermined frequency;
wherein in the first potential embodiment of the disclosure, the predetermined frequency is 500 hz;
wherein in the first potential embodiment of the disclosure, the signal source is generated by a computer.
